(19) United States
(12) Patent Application Publication
(10) Pub. No.: US 2008/0142385 A1
(43) Pub. Date: Jun. 19, 2008

(54) Twist tip cleaning devices for ear wax removal

(76) Inventors: Michael J. Stein, Hampstead, NH (US); Udo H. Fritsch, Chester, NH (US)

Correspondence Address: PEARSON & PEARSON, LLP, 10 GEORGE STREET, LOWELL, MA, 01852, US

(21) Appl. No.: 11/641,302

(22) Filed: Dec. 18, 2006

(51) Int. Cl. A61F 13/38; A61B 19/02

(52) U.S. Cl. 206/362; 604/1

(57) ABSTRACT

Left-handed and right-handed twist tip cleaning devices for cleaning left and right ear canals of a user comprise a shaft having a soft twist tip head which is positioned over a hemisphere type end of the shaft which is supported by two angled inwardly legs. The soft twist tip head has six flutes encircling a bullet shaped body along the longitudinal axis of the shaft. The flutes of the left-handed twist tip cleaning device differ from the right-handed twist tip cleaning device by angling the flutes to optimize cleaning of the ear canals when rotated by the user. A tickler extends from the top of the twist tip head to provide a safety warning when the tickler contacts an ear drum. A storage box comprises a plurality of left-handed twist tip cleaning devices in one-half of the box with one end removably hinged to an edge of the box. A second half of the storage box comprises a plurality of right-handed twist tip cleaning devices removably hinged to an edge of the box.

### BACKGROUND OF THE INVENTION

1. Field of the Invention

This invention relates generally to an apparatus for removal of ear wax or cerumen from an ear canal, and in particular, to a pair of ear cleaning twist tip devices wherein a first device comprises right-handed spiral flutes and a second device comprises left-handed spiral flutes.

2. Description of Related Art

Many devices are described in the prior art for use in removing wax from the outer ear canal. Ear wax or cerumen is a substance produced by the skin of the outer one-third of the ear. It functions to protect the delicate ear canal lining from excessive moisture, dehydration and infection by bacteria and other micro-organisms. Unfortunately, the ear wax can also accumulate fouling hearing aids and other “in-canal” listening devices. The use of cotton swabs, which are well-known, generally pushes the wax deeper into the ear canal mixing it with cotton fibers, dead skin cells and environmental dust forming what doctors call “cerumen impaction” forming hard plugs up to an inch long which can be difficult and painful to remove.

The following patents or patent publications disclose devices intended to be used for cleaning ear canals:

U.S. Pat. No. 3,923,061, issued Dec. 2, 1975 to Jacques Rossignol of France, discloses a tool to extract ear wax having at least one padding on each end of a stick, made up of numerous flexible curved lamellae. Lamellae are individual layers which may be made out of rubber, and appears to be a blunt Q-Tip shaped device. However, the blunt end will tend to force cerumen deeper into the ear canal, which if too hard, can damage ear structures on contact.

U.S. Pat. No. 5,374,276, issued Dec. 20, 1995, to William P. Lay, discloses an ear wax remover comprising a metal rod having an extraction head on a first end and a swab attachment portion on a second end. The extractor head has a generally mushroom shaped tip and a pair of frusto-conical projections extending annularly around the rod adjacent to the tip. However, the mushroom shaped tip will force wax deeper into an ear canal. The second metal end with cotton is likely to become exposed and puncture the fragile ear drum.

U.S. Pat. No. 5,632,756, issued May 27, 1997 to Kenneth Kruglick, discloses an ear cleaning device utilizing a bulbous banded cage with different round shapes made of plastic, having openings on one end of a handle. A collar component controls the depth of insertion of the multiple banded cage into the ear canal. On the other handle end is an assembly for cleaning the ear canal. The cleaning assembly includes a shank extending from the second end of the handle. A cotton swab is on a distal end of the shank. However there is no such thing as a depth stop for ear cleaning devices because hardened cerumen impaction can extend a variable distance from the ear drum right up to the external ear. Pushing hard enough on a wax impaction will damage the ear.

U.S. Pat. No. 6,033,417, issued to Shao-Chien Tseng, discloses a roll-scraping ear pick comprising a stick, a seat, a screw spring set having an exterior screw spring and an interior screw opening. The seat is made of soft plastic and formed to have soft stop ends and neck between ends. The screw spring set mounts on the neck portion and the rings formed by the exterior screw spring can be used to scrape the inner wall of an ear, and the curved exterior screw spring scrapes ear wax. However, the blunt end will force wax deeper into the ear canal and will damage the ear drum and attached middle ear structures on contact. The springs are more likely to abrade the ear canal causing pain, bleeding and possible infection.

U.S. Pat. No. 6,187,021, issued Feb. 13, 2001 to Becker Wim, discloses a portable electric motor driven ear cleaning device comprising a rotary brush comprising a brush element with soft hairs and a brush holder. There are numerous hairs which are “limp” when the brush is not rotating, but the hairs extend outward when the brush is rotated, and the extended soft hairs touch the ear canal engaging the ear wax and thereby cleaning the ear. However, using any type of motor that can generate up to 12,000 RPM without reduction gearing to spin anything in a human auditory canal is not a safe procedure. Also, the softest of bristles will instantly load with wax stiffening and ripping the delicate canal lining to shreds causing severe discomfort, bleeding and possible infection. The lining of the external auditory canal is 100 times more fragile and sensitive than a fingertip.

U.S. Patent Publication No. 2003/0135228, published Jul. 17, 2003 by inventor, Anthony K. Crespo, discloses a disposable ear cleaning appliance comprising an elongated stem in the form of a hollow tube. Within one end of the hollow tube, a cleaning head is secured to enter the ear canal for removal of foreign matter. The head comprises a plurality of lengths of monofilament. Outward ends of the filaments are bonded together, as by sonic welding. A bundle of inboard ends of the several lengths of monofilament is received within the hollow end of the stem and bonded in place by sonic welding. However, this device is similar to an ear curette used by physicians only under direct visual inspection of the ear canal by an otoscope. Such devices are very dangerous because the tip can force cerumen back into the ear canal impacting it against the ear drum. Further, the filament loops that are stiff may cut through the cerumen and into the ear canal wall causing pain and potentially damaging the ear drum and middle ear structure.

### SUMMARY OF THE INVENTION

Accordingly, it is therefore an object of this invention to provide a safe and effective ear cleaning device having an auger shaped tip attached on an end of a shaft.

It is another object of the invention to provide a right-handed twist tip cleaning device and a left-handed twist tip cleaning device for effective cleaning of an ear canal when the right-handed twist tip cleaning device is rotated clockwise in a right ear canal and the left-handed twist tip cleaning device is rotated counterclockwise in a left ear canal.

It is a further object of this invention to provide a soft protrusion or “tickler” on the top of the twist tip cleaning device to contact and protect an ear drum.

It is yet another object of this invention to provide a shaft having on one end a semi-circular loop topped with a mushroom type cap for keeping a soft tip anchored to the shaft and to prevent the twist tip head from spinning or being pulled off or pushed further down on the shaft.

These and other objects are further accomplished by an ear canal cleaning device comprising a shaft having a receiving means extending from an end, a twist tip head positioned on the receiving means on the end of the shaft, and the twist tip head comprises a plurality of flutes encircling the twist tip head at a predetermined angle optimized for cleaning the ear canal when the shaft is rotated. The cleaning device comprises a soft protrusion extending from the twist tip head. The twist tip head comprises a left-handed twist tip head. The left-handed twist tip head comprises the plurality of flutes, the flutes being at an angle in a range of 30 to 60 degrees with respect to a long axis of the shaft encircling the twist tip head. The angle of the flutes includes a preferred angle of approximately 45 degrees. Alternatively, the twist tip head comprises a right-handed twist tip head. The right-handed twist tip head comprises the plurality of flutes, the flutes being at an angle of 30 to 60 degrees with respect to a long axis of the shaft. The angle of the flutes includes a preferred angle of approximately 45 degrees. The twist tip head comprises a rubber having a predetermined hardness. The predetermined hardness of the rubber is in a durometer range of 2-15 on a Shore A scale. The receiving means comprises a hemisphere structure positioned on a pair of support legs, each of the support legs having a first portion extending from the end of the shaft parallel to a long axis of the shaft and a second portion extending from the first portion at a predetermined angle toward the long axis of the shaft.

The objects are further accomplished by a twist tip head comprising a plurality of flutes encircling the head at an angle in the range of 30 to 60 degrees with respect to a center axis of the flute. The twist tip head comprises a soft protrusion extending from an outer end of said head. The twist tip head comprises a left-handed plurality of flutes or a right-handed plurality of flutes. The twist tip head comprises a rubber having a hardness in a durometer range of 2 to 15 on a Shore A scale.

The objects are further accomplished by an enclosure for storing a plurality of twist tip cleaning devices comprising a first section of the enclosure hinged to a second section. The first section includes a plurality of twist tip cleaning devices, each one of the twist tip cleaning devices having an end removably attached to the first section, and the second section includes a plurality of twist tip cleaning devices, each one of the twist tip cleaning devices having an end removably attached to the second section. The first section comprises a plurality of left-handed twist tip cleaning devices and the second section comprises a plurality of right-handed twist tip cleaning devices.

The objects are further accomplished by a box for storing twist tip cleaning devices comprising a first half of the box having a plurality of the twist tip cleaning devices attached to an edge of the first half of the box, the second half of the box having a plurality of the twist tip cleaning devices attached to an edge of the second half of said box, and means for enabling the first half of the box to fold over the second half of the box. The box comprises means for fastening the first half of the box to the second half of the box. The first half of the box comprises a plurality of removable left-handed twist tip cleaning devices. The second half of the box comprises a plurality of removable right-handed twist tip cleaning devices. The box comprises a handle positioned on a side of the box to enable hanging the box on a hook. The attached twist tip cleaning devices are hingedly attached to an edge of the first half and the second half of the box. The second edge of the first half of the box comprises a plurality of notches for receiving a tickler portion of the twist tip devices. The second edge of the second half of the box comprises a plurality of notches for receiving a tickler portion of the twist tip devices.

The objects are further accomplished by a method for providing an ear canal cleaning device comprising the steps of providing a shaft having receiving means extending from an end, positioning a twist tip head on the receiving means on the end of the shaft, and encircling the twist tip head with a plurality of flutes at a predetermined angle optimized for cleaning the ear canal when the shaft is rotated. The method comprises the steps of providing a left-handed twist tip head, with the plurality of flutes encircling the twist tip head at an angle in range of 30 to 60 degrees with respect to a long axis of said shaft, and providing a right-handed twist tip head with a plurality of flutes encircling the twist tip head at an angle in a range of 30 to 60 degrees with respect to a long axis of the shaft. The step of positioning the twist tip head on the receiving means comprises the step of using rubber to make the twist tip head, the rubber having a hardness in a durometer range of 2 to 15 on a Shore A scale.

Additional objects, features and advantages of the invention will become apparent to those skilled in the art upon consideration of the following detailed description of the preferred embodiments exemplifying the best mode of carrying out the invention as presently perceived.

### BRIEF DESCRIPTION OF THE DRAWINGS

The appended claims particularly point out and distinctly claim the subject matter of this invention. The various objects, advantages and novel features of this invention will be more fully apparent from a reading of the following detailed description in conjunction with the accompanying drawings in which like reference numerals refer to like parts, and in which:

FIG. 1A is a front elevational view of a left-handed twist tip cleaning device according to the present invention;

FIG. 1B is a front elevational view of a right-handed twist tip cleaning device according to the present invention;

FIG. 2A is a perspective view of the left-handed twist tip cleaning device;

FIG. 2B is a perspective view of the right-handed twist tip cleaning device;

FIG. 3A is a top plan view of the left-handed twist tip cleaning device;

FIG. 3B is top plan view of the right-handed twist tip cleaning device;

FIG. 4A illustrates a medium size left-handed twist tip cleaning device showing typical dimensions;

FIG. 4B illustrates a medium size right-handed twist tip cleaning device showing typical dimensions;

FIG. 5A is a cross-sectional view of the left-handed twist tip head along line 5A-5A in FIG. 1A;

FIG. 5B is a cross-sectional view of the right-handed twist tip head along line 5B-5B in FIG. 1B;

FIG. 6 is a front elevational view of an end of a shaft prior to attaching a twist tip head;

FIG. 7 is a perspective view of an end of a shaft prior to receiving the twist tip head;

FIG. 8 is a perspective view of a box for storing the twist tip cleaning devices;

FIG. 9 is a front elevational view of the twist tip cleaning device storage box in an open position having left-handed twist tip cleaning devices in a first half and right-handed twist tip cleaning devices in a second half of the box;

FIG. 10 is a side elevational view of the open twist tip storage box of FIG. 9;

FIG. 11 is an enlarged view of the attachment joint where a twist tip cleaning device attaches to the storage box;

FIG. 12 is a perspective view of an open storage box of twist tip cleaning devices with the devices extended away from the box; and

FIG. 13 is a representation of a right-handed twist tip cleaning device inserted within the outer portion of an ear canal.

### DESCRIPTION OF ILLUSTRATIVE EMBODIMENT

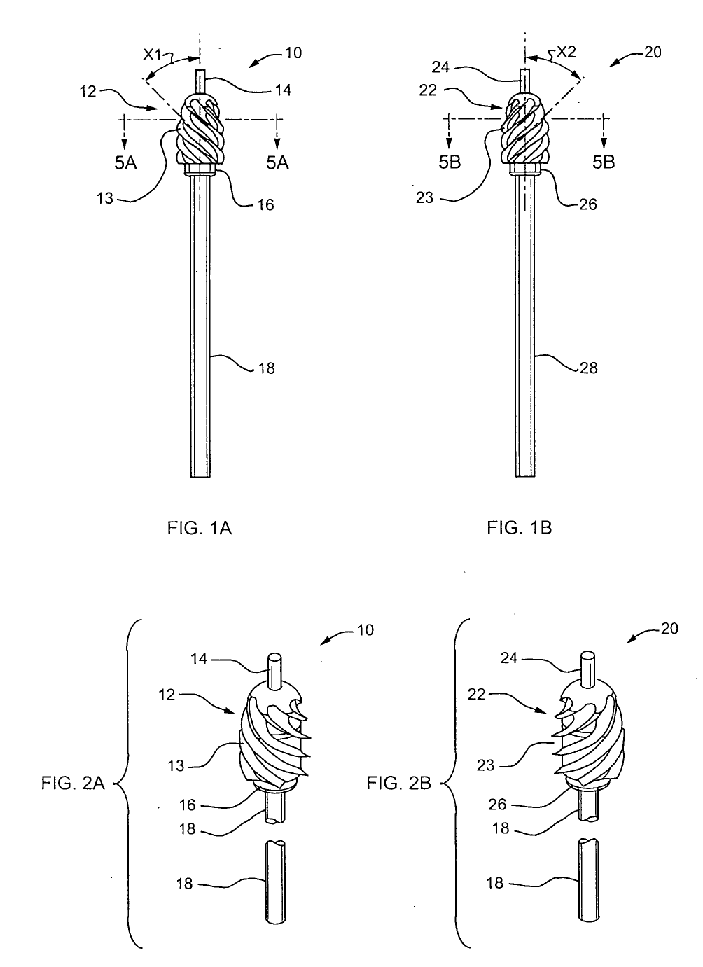

Referring to FIG. 1A and FIG. 1B, FIG. 1A is a front elevational view of a left-handed twist tip ear cleaning device 10 and FIG. 1B is a front elevational view of a right-handed twist tip ear cleaning device 20. Both cleaning devices are used for maintaining hygiene of the external auditory canal by removing cerumen, which is a wax-like substance produced in the outer one-third of the normal external auditory canal. Each of the twist tip cleaning devices 10, 20 comprises a shaft 18, 28 and a soft tip head 12, 22 which is positioned on top of the shaft 18, 28, which is approximately three inches long. The shaft 18, 28 may be made of molded plastic, and the soft tips 12, 22 may be made of very soft molded rubber in the durometer range of “2” to “15” on the Shore A scale as per the ASTM D2240 test standard. If too soft, the flutes 13, 23 compress and do not clean wax, and if too stiff, the ear will be damaged. Extending from the top of each of the soft tips 12, 22 is a protrusion or tickler 14, 24 which in the present embodiment is approximately one millimeter in diameter and two millimeters long. If either one of the twist tip cleaning devices 10, 20 is inserted too far into an ear canal, the tickler 14, 24, which is extremely soft and flexible, contacts the ear drum first thereby warning the user that the twist tip cleaning device has gone far enough into the ear canal.

Referring to FIGS. 1A, 1B, 2A and 2B, FIG. 2A is a perspective view of the left-handed twist tip cleaning device 10 and FIG. 2B is a perspective view of the right-handed twist tip cleaning device 20. Each of the cleaning devices 10, 20 comprises six spiral flutes 13, 23 on a bullet shaped body. The flutes 13, 23 are formed at an angle in the range of 30 degrees to 60 degrees with respect to a long axis of the shafts 18, 28 and a preferred (X1 and Y1) angle is 45 degrees as shown in FIG. 1A and FIG. 1B respectively. The auger shaped twist tip cleaning devices 10, 20 collect the cerumen in the flutes 13, 23 always moving the cerumen outwards by virtue of the rotational action provided by a user. The angle or direction of the spiral flutes distinguishes the left-hand twist tip cleaning device 10 from the right-hand twist tip cleaning device 20 for use in either a left ear or right ear respectively. This is necessary because users of the ear cleaning devices 10, 20 have a natural tendency to twist the shaft 18, 28 between the index finger and the thumb, moving the index finger down the thumb twisting the left-handed twist tip cleaning device 10 counter-clockwise in the left ear and the right-handed twist tip cleaning device 20 clockwise in the right ear.

Referring to FIGS. 1A, 1B, 2A, 2B, 4A, 4B, 5A and 5B, FIG. 4A illustrates a medium size left-handed twist tip cleaning device 10 showing typical dimensions and FIG. 4B illustrates a medium size right-handed twist tip cleaning device 20 showing typical dimensions. FIG. 5A is a cross-sectional view of the left-handed twist tip head 12 along line 5A-5A in FIG. 1A, and FIG. 5B is a cross-sectional view of the right-handed twist tip head 22 along line 5B-5B in FIG. 1B. Typical dimensions of a medium size twist tip are indicated in FIGS. 5A and 5B. Smaller and larger tips may be molded on the same shafts for people with smaller (women) or larger (tall men) ear canals. For example, the diameter D3 of the medium size twist tip head is FIG. 5A is 0.186 inches. For a small size twist tip D3 is 0.150 inches and for a large size, D3 is 0.200 inches.

In FIG. 4A, the preferred dimensions for a medium size left-handed twist tip cleaning device 10 are indicated and the approximate dimensions (in inches) are as follows:

| | |
|---|---|
| L1 | 2.190 |
| L2 | 0.125 |
| L3 | 0.375 |
| L4 | 0.063 |
| L5 | 0.187 |
| L6 | 0.100 |

In FIG. 4B, the preferred dimensions for a medium size right-handed twist tip cleaning device 20 are indicated and the approximate dimensions (in inches) are as follows:

| | |
|---|---|
| L10 | 2.190 |
| L11 | 0.125 |
| L12 | 0.375 |
| L13 | 0.063 |
| L14 | 0.187 |
| L15 | 0.100 |

In FIG. 5A, the preferred dimensions including radius for a medium left-handed twist tip head 12 are indicated and the approximate dimensions (in inches) are as follows:

| | | | |
|---|---|---|---|
| D1 | 0.068 | R1 | 0.162 |
| D2 | 0.032 | R2 | 0.162 |
| D3 | 0.186 | | |
| D4 | 0.160 | | |
| D5 | 0.069 | | |
| D6 | 0.022 | | |

In FIG. 5B, the preferred dimensions including radius for a medium right-handed twist tip head 22 are indicated and the approximate dimensions (in inches) are as follows:

| | | | |
|---|---|---|---|
| D10 | 0.068 | R10 | 0.162 |
| D11 | 0.032 | R11 | 0.162 |
| D12 | 0.186 | | |
| D13 | 0.160 | | |
| D14 | 0.069 | | |
| D15 | 0.022 | | |

For small size and large size twist tips, the diameter dimensions D3 and D12 become approximately 0.150 inches for a small size twist tip and approximately 0.200 inches for a large size twist tip. One of ordinary skill in the art will recognize that other dimensions may be implemented to make small, medium and large size twist tips both left-handed and right-handed for ear canal cleaning.

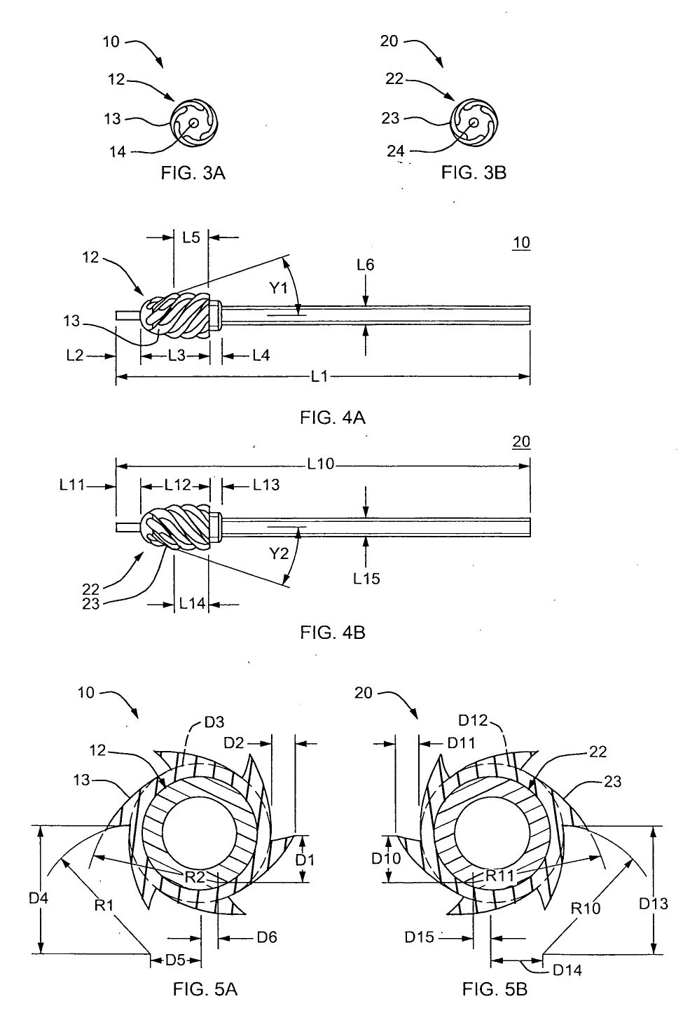

Referring to FIGS. 3A and 3B, FIG. 3A is a top plan view of the left-handed twist tip cleaning device 10, and FIG. 3B is a top plan view of the right-handed twist tip cleaning device 20.

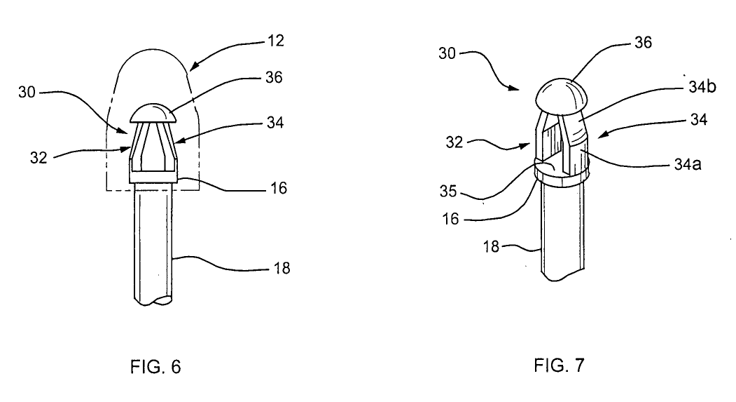

Referring now to FIG. 6 and FIG. 7, FIG. 6 is a front elevational view of an end 30 of the shaft 18 prior to attaching the twist tip head 12, and FIG. 7 is a perspective view of the end 30 of the shaft 18 constructed for receiving and retaining the twist tip head 12. The end 30 of shaft 18 comprises a mushroom type cap or hemisphere 36 positioned on top of two support legs 32, 34 each having a section 34a rising vertically and then having a section 34b angled inward for contacting the bottom of the hemisphere. The support legs 34, 36 rest on a flat area 35 of the shaft end 30. The mushroom type cap or hemisphere 36 is provided to keep the twist tip head 12 anchored to the shaft 18, and to prevent the twist tip head 12 from spinning or being pulled off or pushed down on the shaft 18.

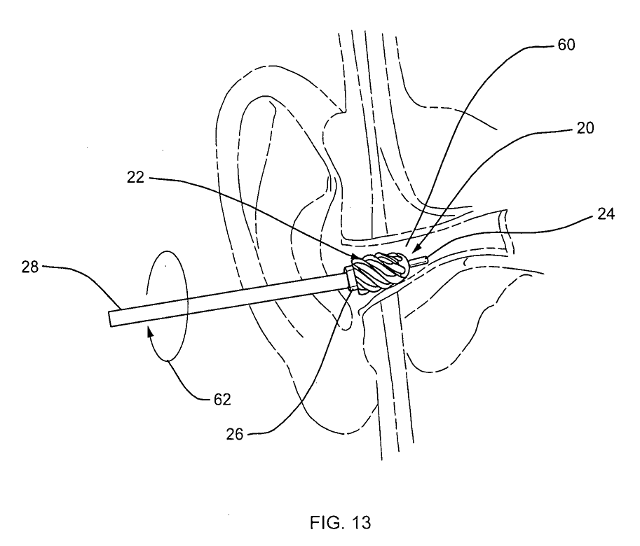

Referring to FIG. 13 an illustration is provided of a right-handed twist tip cleaning device 20 inserted within the outer portion of an ear canal 60 to be rotated clockwise 62 by a user.

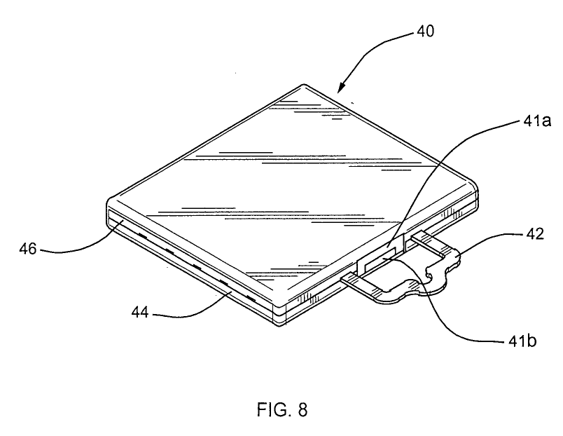

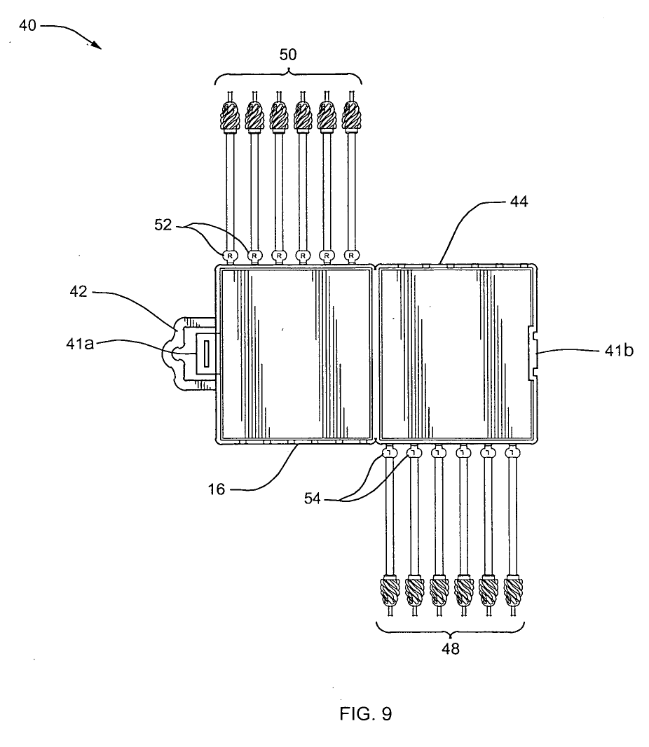

Referring to FIG. 8 and FIG. 9, FIG. 8 is a perspective view of a box 40 for storing the twist tip cleaning devices 10, 20, and FIG. 9 is a front elevational view of the twist tip cleaning device storage box 40 in an open position having left-handed twist tip cleaning devices 10 in a first half 44 of the storage box 40 and right-handed twist tip cleaning devices 20 in a second half 46 of the storage box 40. The storage box 40 comprises a handle 42 for carrying or hanging purposes and a snap catch 41a and 41b for securing the two halves 44, 46 in a closed position. An insert 41b is included on the end of the first half 44 of the storage box 40 and a receiving tab 41a is included on a corresponding end of the second half 46 of the storage box 40 and located within the opening formed by the handle 42.

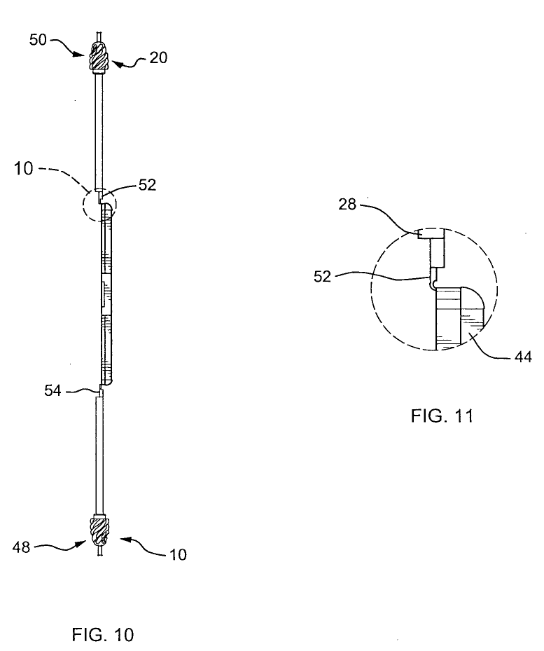

Referring to FIGS. 9, 10, and 11, FIG. 10 is a side elevational view of the open twist tip storage box 40 of FIG. 9 showing an attachment joint or hinge 52 connecting one of the plurality of right-handed twist tip cleaning devices 50 to the storage box 40 and a hinge 54 connecting one of the plurality of left-handed twist tip cleaning devices 48 to the box. FIG. 9 shows each of the twist tip cleaning devices in groups 48 and 50 similarly attached to the box by hinges 52, 54. FIG. 11 is an enlarged view of the attachment joint or hinge 52 connecting an end of a shaft 28 to the first half of the storage box 40. The hinge 52 is very thin at the point of connection with the storage box 40 enabling the shaft 28 to be easily removed from the storage box 40. The hinges 52, 54 are cast into the plastic during the injection molding of the case using machines and techniques well known in the plastic industry. The case and shafts are injection molded at a first station in an automated two station injection molding machine. The tips are made of an entirely different material and are then injection molded onto the shafts at the second station.

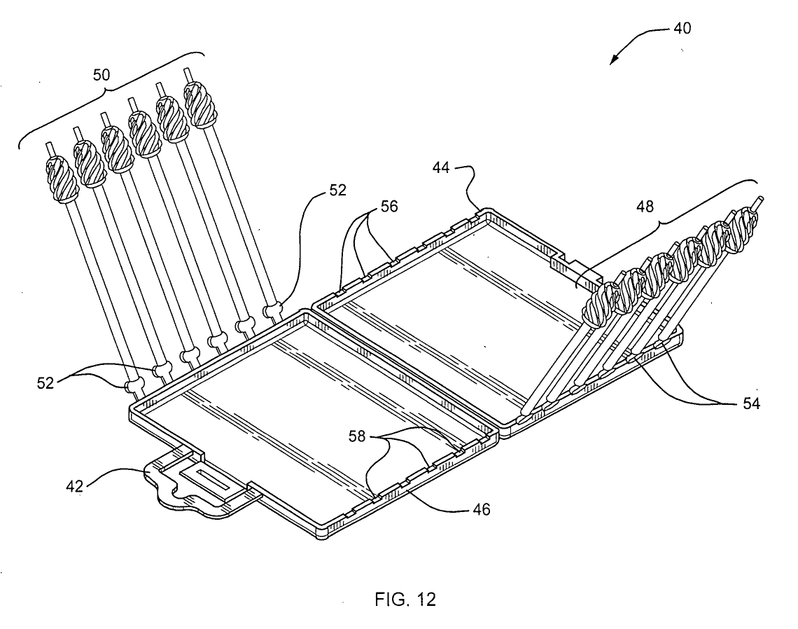

Referring now to FIG. 12, FIG. 12 is a perspective view of the open storage box 40 of a plurality of twist tip cleaning devices 48, 50 showing the cleaning devices 48, 50 extending away from the storage box 40. When the plurality of twist tip cleaning devices 48, 50 are placed within each half 44, 46 of the storage box 40 so that the storage box 40 may be closed, notches 56, 58 along an edge of each half 44, 46 and on an opposite side from the hinges 52, 54 provide an opening for the tickler 14, 24 portion of twist tip heads 12 and 22 to rest.

This invention has been disclosed in terms of certain embodiments including a left-handed twist tip cleaning device 10 and a right-handed twist tip cleaning device 20 and a box 40 for storing the twist tip cleaning devices. It will be apparent that many modifications can be made to the disclosed apparatus without departing from the invention. Therefore, it is the intent of the appended claims to cover all such variations and modifications as come within the true spirit and scope of this invention.

What is claimed is:

1. An ear canal cleaning device comprising:
a shaft having a receiving means extending from an end;
a twist tip head positioned on said receiving means on said end of said shaft; and
said twist tip head comprises a plurality of flutes encircling said twist tip head at a predetermined angle optimized for cleaning said ear canal when said shaft is rotated.

2. The ear canal cleaning device as recited in claim 1 wherein said cleaning device comprises a soft protrusion extending from said twist tip head.

3. The ear canal cleaning device as recited in claim 1 wherein said twist tip head comprises a left-handed twist tip head.

4. The ear canal cleaning device as recited in claim 3 wherein said left-handed twist tip head comprises said plurality of flutes, said flutes being at an angle in a range of 30 to 60 degrees with respect to a long axis of the said shaft encircling said twist tip head.

5. The ear canal cleaning device as recited in claim 4 wherein said angle of said flutes includes a preferred angle of approximately 45 degrees.

6. The ear canal cleaning device as recited in claim 1 wherein said twist tip head comprises a right-handed twist tip head.

7. The ear canal cleaning devices as recited in claim 6 wherein said right-handed twist tip head comprises said plurality of flutes, said flutes being at an angle of 30 to 60 degrees with respect to a long axis of the said shaft.

8. The ear canal cleaning device as recited in claim 7 wherein said angle of said flutes includes a preferred angle of approximately 45 degrees.

9. The ear canal cleaning device as recited in claim 1 wherein said twist tip head comprises a rubber having a predetermined hardness.

10. The ear canal cleaning device as recited in claim 9 wherein said predetermined hardness of said rubber is in a durometer range of 2-15 on a Shore A scale.

11. The ear canal cleaning device as recited in claim 1 wherein said receiving means comprises a hemisphere structure positioned on a pair of support legs, each of said support legs having a first portion extending from said end of said shaft parallel to a long axis of said shaft and a second portion extending from said first portion at a predetermined angle toward said long axis of said shaft.

12. A twist tip head comprising a plurality of flutes encircling said head at an angle in the range of 30 to 60 degrees with respect to a center axis of said flute.

13. The twist tip head as recited in claim 12, wherein said head comprises a soft protrusion extending from an outer end of said head.

14. The twist tip head as recited in claim 12 comprises a left-handed plurality of flutes.

15. The twist tip head as recited in claim 12 comprises a right-handed plurality of flutes.

16. The twist tip head as recited in claim 12 comprises a rubber having a hardness in a durometer range of 2 to 15 on a Shore A scale.

17. An enclosure for storing a plurality of twist tip cleaning devices comprising:
a first section of said enclosure hinged to a second section;
said first section includes a plurality of twist tip cleaning devices, each one of said twist tip cleaning devices having an end removably attached to said first section; and
said second section includes a plurality of twist tip cleaning devices, each one of said twist tip cleaning devices having an end removably attached to said second section.

18. The enclosure as recited in claim 17 wherein said first section comprises a plurality of left-handed twist tip cleaning devices.

19. The enclosures as recited in claim 17 wherein said second section comprises a plurality of right-handed twist tip cleaning devices.

20. A box for storing twist tip cleaning devices comprising:
a first half of said box having a plurality of said twist tip cleaning devices attached to an edge of said first half of said box;
a second half of said box having a plurality of said twist tip cleaning devices attached to an edge of said second half of said box;
means for enabling said first half of said box to fold over said second half of said box;

21. The box as recited in claim 20 wherein said box comprises means for fastening said first half of said box to said second half of said box.

22. The box as recited in claim 20 wherein said first half of said box comprises a plurality of removable left-handed twist tip cleaning devices;

23. The box as recited in claim 20 wherein said second half of said box comprises a plurality of removable right-handed twist tip cleaning devices.

24. The box as recited in claim 20 wherein said box comprises a handle positioned on a side of said box to enable hanging said box on a hook.

25. The box as recited in claim 20 wherein said attached twist tip cleaning devices are hingedly attached to an edge of said first half and said second half of said box.

26. The box as recited in claim 20 wherein a second edge of said first half of said box comprises a plurality of notches for receiving a tickler portion of said twist tip devices;

27. The box as recited in claim 20 wherein a second edge of said second half of said box comprises a plurality of notches for receiving a tickler portion of said twist tip devices;

28. A method for providing an ear canal cleaning device comprising:
providing a shaft having receiving means extending from an end;
positioning a twist tip head on said receiving means on said end of said shaft;
encircling said twist tip head with a plurality of flutes at a predetermined angle optimized for cleaning said ear canal when said shaft is rotated.

29. The method as recited in claim 28 wherein said method comprises the step of providing a soft protrusion extending from said twist tip head.

30. The method as recited in claim 28 wherein said method comprises the step of providing a left-handed twist tip head.

31. The method as recited in claim 30 wherein said step of providing a left-handed twist tip head comprises the step of said plurality of flutes encircling said twist tip head at an angle in a range of 30 to 60 degrees with respect to a long axis of said shaft.

32. The method as recited in claim 28 wherein said method comprises the step of providing a right-handed twist tip head.

33. The method as recited in claim 32 wherein said step of providing a right-handed twist tip head comprises the step of said plurality of flutes encircling said twist tip head at an angle in a range of 30 to 60 degrees with respect to a long axis of said shaft.

34. The method as recited in claim 28 wherein said step of positioning said twist tip head on said receiving means comprises the step of using rubber to make said twist tip head, said rubber having a hardness in a durometer range of 2 to 15 on a Shore A scale.
